(19) United States
(12) Patent Application Publication
(10) Pub. No.: US 2015/0190450 A1
(43) Pub. Date: Jul. 9, 2015

(54) INGREDIENT FOR CONSUMPTION AND APPLICATION

(71) Applicant: Alice Chang, South Pasadena, CA (US)

(72) Inventor: Alice Chang, South Pasadena, CA (US)

(21) Appl. No.: 14/159,014

(22) Filed: Jan. 20, 2014

Related U.S. Application Data
(60) Provisional application No. 61/923,707, filed on Jan. 5, 2014.

(51) Int. Cl. A61K 36/84; A61Q 11/00; A61Q 17/04; A61K 8/97; A61K 36/258; A61K 36/752; A61K 8/98; A61K 9/70; A61Q 15/00

(52) U.S. Cl. CPC A61K 36/84; A61K 9/7007; A61Q 11/00; A61Q 17/04; A61Q 15/00; A61K 36/258; A61K 36/752; A61K 8/987; A61K 8/97

(57) ABSTRACT

Ingredient for consumption and application is created in various methods and forms so that the ingredient is available for the consumer in a convenient, effective, and efficient manner. The ingredient includes plant ingredient, chinese medicine, nutritional ingredient, health ingredient, cleansing ingredient, and food additive. Ingredient for consumption and application comprising at least one ingredient. The ingredient having nutrients including carbohydrates, vitamins, minerals, and antioxidants; having medicinal properties; and having cleansing properties. The ingredient is in form selected from group consisting of skin, skinned, unskinned, unshelled, cut, uncut, extract, juice, sauce, liquid, oil, gel, jelly, wax, cream, paste, powder, thin layer, small particles, small and large pieces. The ingredient can be uncooked; and cooked by method selected from group consisting of deep frying, sun drying, roasting, baking, steaming, boiling, grilling, and frying. The ingredient can be added to various fabric and medium.

### CROSS-REFERENCE TO RELATED APPLICATION

The present application is related to and claims priority from prior provisional application Ser. No. 61/923,707 filed Jan. 5, 2014 which application is incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

1. Field of the Invention

The present invention relates generally to the field of comestibles, and more specifically relates to ingredient for consumption and application in various methods and forms. The ingredient includes plant ingredient, chinese medicine, nutritional ingredient, health ingredient, cleansing ingredient, and food additive.

2. Description of the Related Art

Currently on the market, ingredients including plant ingredient, chinese medicine, nutritional ingredient, health ingredient, cleansing ingredient, and food additive are available in limited methods for consumption and application.

Various attempts have been made to solve the above-mentioned problems such as those found in U.S. Pat. Nos. 6,770,263, 8,362,034, 2002/0031509, 2008/0311230, 2013/0171263, 2013/0287870. This prior art is representative of comestibles relating to food. None of the above inventions and patents, taken either singly or in combination, is seen to describe the invention as claimed.

Ideally, more convenient, effective, and efficient methods are needed to deliver the ingredients for consumption and application. And yet manufactured at a modest expense.

### BRIEF SUMMARY OF THE INVENTION

In view of the foregoing disadvantages inherent in the known related comestibles art, the present invention provides ingredients for consumption and application in various methods and forms, so that the ingredients are available for the consumer in a convenient, effective, and efficient manner. The ingredient include plant ingredient, chinese medicine, nutritional ingredient, health ingredient, cleansing ingredient, and food additive.

Ingredient for consumption and application is created comprising at least one ingredient.

The present invention holds significant improvements and serves as ingredients for consumption and application in various methods and forms, so that the ingredients are available for the consumer in a convenient, effective, and efficient manner. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### DETAILED DESCRIPTION

As discussed above, embodiments of the present invention relate to comestibles and more particularly to ingredients for consumption and application in various methods and forms, so that the ingredients are available for the consumer in a convenient, effective, and efficient manner. The ingredient include plant ingredient, chinese medicine, nutritional ingredient, health ingredient, cleansing ingredient, and food additive.

As mentioned earlier, ingredient for consumption and application is created comprising at least one ingredient.

At least one ingredient having nutrients including carbohydrates, vitamins, minerals, and antioxidants. At least one ingredient having medicinal properties. At least one ingredient having cleansing properties. At least one ingredient is in form selected from group consisting of skin, skinned, unskinned, unshelled, cut, uncut, extract, juice, sauce, liquid, oil, gel, jelly, wax, cream, paste, powder, thin layer, small particles, small pieces, and large pieces. At least one ingredient can be uncooked. At least one ingredient can be cooked by method selected from group consisting of deep frying, sun drying, roasting, baking, steaming, boiling, grilling, and frying. At least one ingredient can be added to various fabric including waterproof fabric, synthetic fabric, and natural fabric. At least one ingredient can be added to various medium including solid, liquid, gas, and vacuum. At least one ingredient can be added to various fabric by method selected from group consisting of imbedded, coated, sewn, and glued.

At least one ingredient for consumption can be delivered in form selected from group consisting of ingredient particles, ingredient bag, ingredient dried pieces, as mixed in for food and drink, as topping for food and drink, as mixed in for condiment, as mixed in for organic matter, as coating for organic matter, as mixed in for inorganic matter, and as coating for inorganic matter.

At least one ingredient for application can be delivered in form selected from group consisting of clothing, covering, porous container, candle, fragrance, cosmetics, cleanser, moisturizer, lotion, cream, gel, foam, ointment, wax, oil, sunscreen, bath oil, nasal spray, astringent, face mask, deodorant, antiperspirant, talcum powder, contraceptive, condom, contraceptive sponge, sponge, hair brush, face brush, body brush, dental floss, mouthwash, toothpaste, toothbrush, shampoo, conditioner, soap, detergent, tissue paper, sanitary pad, sanitary napkin, panty liner, tampon, incontinence pad, diaper, underarm liner, sofa, chair, mattress, pillow, cushion, clothes hanger, toy, ornament, jewelry, musical instrument, photo frame, picture frame, vase, flower pot, glassware, dinnerware, tableware, cookware, furniture, car seat, motorcycle seat, bike seat, cleaning device, medical device, wheeled device, exercise equipment, toilet, sink, bathtub, shower, sauna, countertop, kitchen appliance, and home appliance.

At least one ingredient comprising at least one plant ingredient selected from group consisting of fruit, plant, plant oil, herb, tea, root, flower, flower bud, pollen, leaf, branch, bark, stem, stem shoot, bulb, nut, seed, bean, cereal grain, fruit skin, vegetable skin, vegetable, vegetable oil, and vegetable fat, including hop, menthol, cordyceps, echinacea, elder, horehound, mallow, sage, thyme, cowslip, burnet, yarrow, marsh mallow, lady's mantle, speedwell, plantain, linden flower, wild thyme, hyssop, eucalyptus, sea buckthorn, elder blossom, verbena, lemon balm, clove, coriander, cumin, basil, dill, rosemary, parsley, fennel, oregano, allspice, catnip, wormwood, musk, chili, horseradish, turmeric, mustard, curry, wasabi, mountain mint, peppermint, spearmint, wintergreen, cinnamon, licorice, rose, jasmine, chamomile, chrysanthemum, lavender, acacia, safflower, vanilla, asafoetida, balsam, bay, buchu, birch, camphor, cannabis, caraway, calamus, cistus, citronella, clary sage, costus, cubeb, cypress, cypriol, davana, elecampane, fenugreek, fir, frankincense, galbanum, geranium, goldenrod, henna, helichrysum, hickory, juniper, ledum, litsea cubeba, marjoram, melaleuca, mountain savory, mugwort, myrrh, myrtle, neroli, nutmeg, orris, palo santo, petitgrain, pine, ravensara, red cedar, rosewood, sage, sassafras, schisandra, spikenard, spruce, tea tree, tsuga, valerian, yarrow, ylang ylang, zedoary, benzoin, copaiba, manuka, bergamot, patchouli, lilium, ambergris, garenia, oakmoss, labdanum, coumarin, iris, water lily, ivy, amber, carnation, vetiver, cardamom, freesia, cyclamen, muguet, gardenia, tuberose, tonka bean, agarwood, cedarwood, sandalwood, alfafa, apple, orange, lemon, lime, grapefruit, grape, guava, plum, peach, pear, blackberry, blueberry, strawberry, cranberry, raspberry, cherry, mango, pineapple, pomegranate, papaya, watermelon, rockmelon, hamimelon, melon, honeydew, coconut, durian, passion fruit, banana, fig, apricot, mandarin, blackcurrant, starfruit, kiwi, pomelo, lychee, loquat, persimmon, tangerine, mangosteen, noni, sugar apple, mulberry, elderberry, gooseberry, lingonberry, juniper berry, jackfruit, dragonfruit, abiu, acai, acerola, ackee, cherry orange, moringa, ambarella, araza, arhat, atemoya, atherton raspberry, babaco, bacupari, bacuri, bael, barbadine, beach plum, batuan, bearberry, bignay, bilberry, bilimbi, biriba, black apple, black cherry, black raspberry, black mulberry, black sapote, blood orange, bolwarra, boquila, blue tongue, breadfruit, broad leaf bramble, buddha's hand, buffaloberry, brush cherry, burdekin plum, bush tomato, button mangosteen, caimito, calabash tree, calamondin, calamansi, camucamu, canary melon, canistel, cape gooseberry, capuli cherry, carambola, cardon, carob, cashew apple, cassabanana, cattley guava, cawesh, cedar bay cherry, ceriman, cereus peruvianus, charichuelo, chayote, che, chempedak, chenet, cherimoya, cherry ballart, chokeberry, chokecherry, chupa chupa, citron, clementine, cloudberry, cluster fig, cocky apple, coco plum, cocona, cocoplum, coffee cherry, common apple berry, conkerberry, cornelian cherry, crowberry, cupuacu, currant, curry leaf apple, custard apple, elephant apple, key apple, damson plum, date, date plum, davidson's plum, dead man's finger, desert banana, desert fig, desert lime, dodder laurel, doubah, double coconut, duku, eastern may hawthorn, emu apple, emu berry, false mastic, feijoa, fibrous satinash, finger lime, florida strangler fig, gac, galia melon, gambooge, genip, giant granadilla, otaheite gooseberry, goumi, governor's plum, montessa granadilla, yellow granadilla, greengage, grumichama, guanabana, guarana, cattley guava, pineapple guava, purple guava, strawberry guava, guavaberry, hackberry, hairless rambutan, hardy kiwi, hawthorn, hog plum, honeysuckle, horned melon, huckleberry, huito, ice cream bean, llama, lllawarra plum, imbe, jabuticaba, jambul, jatoba, jelly palm, jocote, jujube, kabosu, kaffir lime, kahikatea, kakadu lime, kakadu plum, kapok, karkalla, karonda, kei apple, kepel fruit, keule, key lime, kitembilla, korlan, kumquat, kutjera, kwai muk, lady apple, lakoocha, langsat, lanzone, lapsi, lardizabala, lemon aspen, leucaena, lillypilly, little gooseberry tree, limequat, loganberry, longan, lucuma, ma praang, mabolo, madrono, malabar plum, mamey sapote, mamoncillo, mangaba, manoao, maqui, marang, manila, mayapple, malay apple, mammee apple, maypop, medlar, melinjo, melon pear, midyim, miracle fruit, mock strawberry, purple mombin, red mombin, yellow mombin, monstera, mora de castilla, morinda, morinda citrifolia, mortiño, mountain pepper, mountain soursop, wild plum, mundu, muntries, muscadine, muskmelon, naartjie, nageia, nance, nannyberry, naranjilla, neem, nere, nonda plum, nungu, oil palm, oregan grape, oroblanco, otaheite gooseberry, palmyra palm, pawpaw, peachpalm, peanut butter fruit, pepino, pequi, peumo, phalsa, pigeon pea, pigeon plum, pigface, pindo palm, pineapple guava, pitomba, pois doux, pommecythere, pommerac, prickly pear, prune, pulasan, pummelo, pupunha, purple apple berry, purple granadilla, quandong, queensland ebony, quince, raisin tree, rambutan, red granadilla, red mulberry, rhubarb, riberry, rimu, rollinia, rose apple, rose hip, rose leaf bramble, rose myrtle, rowan, safou, sageretia, saguaro, salak, salal, salmonberry, sandpaper fig, santol, mamey, sapodilla, fibrous satinash, saskatoonberry, saw palmetto, sea buckthorn, serviceberry, shipova, small leaf tamarind, snow berry, soncoya, servicetree, soursop, southern crabapple, spanish lime, spanish tamarind, spiny monkey orange, star apple, carambola, strawberry guava, ugni, strawberry pear, surinam cherry, sweet apple berry, sweet ganadilla, sweet lemon, sweet orange, sweet pepper, sweetsop, sycamore fig, tamarillo, tamarind, monkey tamarind, velvet tamarind, tanjong, texas persimmon, thimbleberry, totara, toyon, wampee, water apple, wax apple, wax jambu, acerola, white aspen, white mulberry, white sapote, wild grape, wild orange, wild peach, wild plum, wild mangosteen, wineberry, wolfberry, wongi, wood apple, yangmei, yellow plum, yew, youngberry, zig zag vine, ahipa, arracacha, bamboo shoot, beetroot, burdock, broadleaf arrowhead, camas, canna, carrot, cassava, celeriac, chinese artichoke, daikon, earthnut pea, elephant foot yam, ensete, galangal, ginger, ginseng, hamberg parsley, lotus root, jerusalem artichoke, jicama, mashua, parsley root, parsnip, pignut, potato, prairie turnip, radish, rutabaga, salsify, scorzonera, skirret, swede, sweet potato, taro, ti, tigernut, turnip, ulluco, wasabi, water caltrop, water chestnut, yalcon, yam, avocado, bell pepper, bitter melon, bitter gourd, breadfruit, chayote, cucumber, ivy gourd, eggplant, aubergine, brinjal, luffa, nopal, okra, pepper, pumpkin, squash, sweet corn, sweet pepper, tinda, tomatillo, tomato, west indian gherkin, winter melon, zucchini, courgette, artichoke, broccoli, caper, cauliflower, courgette flower, globe artichoke, squash blossom, asparagus, prussian asparagus, bamboo shoot, celery, leek, wild leek, lemongrass, rhubarb, cardoon, kurrat, galangal, amaranth, arugula, beet green, bok choy, borage, broccoli rabe, brussel sprout, cabbage, catsear, celery, celtuce, chard, chaya, chickweed, chicory, chinese mallow, chive, choi sum, collard green, common purslane, corn salad, cress, dandelion, endive, fat hen, fiddlehead, fluted pumpkin, garden rocket, golden samphire, good king henry, greater plantain, kai lan, kale, komatsuna, kuka, lagos bologi, lamb's lettuce, lamb's quarters, land cress, leek, lettuce, lizard's tail, malabar spinach, melokhia, miner's lettuce, mizuna green, mustard green, napa cabbage, new zealand spinach, orache, pak choy, paracress, pea, poke, radicchio, samphire, sculpit, stridolo, sea beet, sea kale, sierra leoni bologi, soko, sorrel, spinach, summer purslane, swiss chard, tatsoi, turnip green, watercress, water spinach, wheatgrass, winter purslane, yarrow, yao choy, cardoon, chive, florence fennel, elephant garlic, garlic, garlic chive, kohlrabi, onion, pearl onion, potato onion, spring onion, tree onion, welsh onion, quamash, scallion, shallot, rice, barley, oat, barley, rye, corn, wheat, buckwheat, millet, sorghum, spelt, quinoa, chicory, molasses, beet root, fonio, palmer's grass, teff, triticale, cattail, chia, flax, grain amaranth, canihua, pitseed goosefoot, wattleseed, almond, cashew, chestnut, coconut, hazelnut, macadamia, peanut, pecan, pine nut, pistachio, walnut, acorn, brazil nut, gingko nut, shea nut, beechnut, candlenut, kurrajong, mongongo, palm nut, karuka, red bopple nut, hickory, jacknut, pepea nut, yellow walnut, cycad, munya nut, monkey puzzle nut, paradise nut, colocynth, filbert, mamoncillo, maya nut, ogbono nut, water caltrop, juniper, podocarp, cempedak, coffee, egusi, fox nut, jackfruit, malabar gourd, nutmeg, breadnut, betel nut, cola nut, pili nut, olive, drumstick, okra, pumpkin seed, sesame seed, sunflower seed, lotus seed, hemp seed, green bean, red bean, azuki bean, black bean, back eyed bean, white bean, yellow bean, common bean, broad bean, pinto bean, soybean, lima bean, kidney bean, pea bean, potato bean, dolichos bean, moth bean, mung bean, ricebean, runner bean, tepary bean, urad bean, velvet bean, winged bean, yardlong bean, chickpea, pigeon pea, snap pea, snow pea, lentil, lupin, tarwi, carob, guar, horse gram, cocoa bean, coffee bean, bambara groundnut, cowpea, hyacinth bean, lablab, moringa oleifera, sterculia, yam bean, alfalfa, clover, mesquite, carob, tamarind, wisteria, aonori, arame, carola, caulerpa, dabberlocks, dulse, hijiki, kombu, laver, mozuku, nori, ogonori, sea grape, sea lettuce, sea weed, wakame, truffle, mushroom, bean sprout, acai, aloe vera, arnica, ashoka, asthma weed, astragalus, barberry, belladonna, bitter leaf, bitter orange, black cohosh, blessed thistle, cat's claw, cayenne, chaparral, chasteberry, cinchona, coffee senna, comfrey, digitalis, dong quai, ephedra, european mistletoe, evening primrose, fenugreek, feverfew, flaxseed, gingko, goldenseal, hoodia, horse chestnut, horsetail, jamaica dogwood, jojoba, kava, khat, konjac, kratom, kanna, licorice root, marigold, moringa oleifera, milk thistle, neem, opium poppy, purple coneflower, passion flower, red clover, syrian rue, st john's wort, saw palmetto, tea tree oil, thunder god vine, tulasi, turmeric, umckaloabo, valerian, white willow, yerba santa, ajwain, akudjura, alexanders, alkanet, alligator pepper, angelica, anise, anise hyssop, aniseed myrtle, annatto, apple mint, artemisia, asafoetida, asarabacca, avens, avocado leaf, barberry, bay leaf, bee balm, boldo, borage, black cardamom, blue fenugreek, caraway, cardamom, cassia, cayenne pepper, celery leaf, celery seed, chervil, chicory, cicely, cilantro, clary, costmary, cubeb pepper, cudweed, culantro, curry leaf, curry plant, dill seed, epazote, fenugreek, filé powder, fingerroot, galangal, galingale, ganoderma, golpar, grains of paradise, grains of selim, houttuynia cordata, huacatay, indonesian bay leaf, jimbu, kaffir lime leaves, kala zeera, kawakawa seeds, kencur, keluak, kinh gioi, kokam seed, korarima, koseret leaves, lemon myrtle, lemon ironbark, lemon verbena, leptotes bicolor, lesser calamint, lime flower, lovage, mahlab, marjoram, mastic, mountain horopito, musk mallow, nasturtium, nigella, njangsa, neem, olida, orris root, pandan flower, pandan leaf, paprika, paracress, peppermint gum leaf, perilla, pandanus amaryllifolius, quassia, ramsons, rice paddy herb, rue, saffron, salad burnet, salep, sassafras, savory, silphium, shiso, sorrel, spikenard, star anise, sumac, sweet woodruff, tarragon, voatsiperifery, water-pepper, wattleseed, wild betel, willow herb, wood avens, woodruff, abies balsamea,abronia fragrans, acer glabrum var. douglasii, acer glabrum var. glabrum, acer negundo, acer saccharinum, acer saccharum, actaea racemosa, actaea rubra, agrimonia gryposepala, alnus rhombifolia, alnus rubra, artemisia douglasiana, artemisia ludoviciana, arundinaria, asarum canadense, asclepias verticillata, baccharis sarothroides, balsamorhiza sagittata, baptisia australis, betula occidentalis, blephilia ciliata, bloodroot, calypso, cardamine diphylla, culophyllum, canothus inintegerrimus, canothus velutinus, cimaphila umbellata, caytonia virginica, ceome serrulata, cmmelina dianthifolia, cornus sericea, delpinium nudicaule, devil's club, echinacea, encelia farinosa, ephedra californica, equisetum hyemale, erigenia bulbosa, eriodictyon crassifolium, eryngium aquaticum, erythrina herbacea, eurybia macrophylla, gaultheria hispidula, gaultheria procumbens, gentiana villosa, geranium maculatum, goldenseal, gutierrezia microcephala, hamamelis virginiana, heracleum maximum, holodiscus discolor, holodiscus dumosus, hydrangea arborescens, hydrangea cinerea, ilex verticillata, iris missouriensis, jeffersonia diphylla, juniperus communis, juniperus scopulorum, krascheninnikovia lanata, larrea tridentata, lobelia, mahonia aquifolium, mahonia nervosa, mahonia repens, malosma, menispermum canadense, osmunda claytoniana, pectis papposa, persicaria amphibia, pinus strobus, pluchea sericea, podophyllum peltatum, populus tremuloides, prunus emarginata, prunus virginiana, ribes aureum, ribes divaricatum, ribes glandulosum, ribes laxiflorum, ribes oxyacanthoides, salvia apiana, salvia mellifera, trichostema lanatum, trichostema lanceolatum, umbellularia, viburnum prunifolium, virginia iris, yucca glauca, agaricus bisporus, agaricus subrufescens, agrocybe aegerita, auricularia auricula, auricularia polytricha, boletus badius, boletus edulis, coprinus comatus, cordyceps sinensis, flammulina velutipes, grifola frondosa, hypsizygus tessellatus, lentinula edodes, lignosus rhinocerus, morchella esculenta, phallus impudicus, phallus indusiatus, pholiota nameko, pleurotus citrinopileatus, pleurotus djamor, pleurotus eryngii, pleurotus ostreatus, sparassis crispa, tremella fuciformis, tremella mesenterica, tricholoma matsutake, ustilago maydis, volvariella volvacea, astraeus hygrometricus, cyathus stercoreus, cyathus striatus, fomes fomentarius, ganoderma lucidum, hydnellum peckii, inonotus obliquus, peziza vesiculosa, phellinus linteus, piptoporus betulinus, polyporus umbellatus, poria cocos, schizophyllum commune, taiwanofungus camphoratus, trametes gibbosa, trametes versicolor, xylaria hypoxylon, fungus, and mold.

At least one ingredient comprising at least one chinese medicine selected from group consisting of snake, snake venom, gecko, scorpion, seahorse, beetle, centipede, leech, fungus, cammellia, cayenne, chinese cucumber, cinchona tree, xanthium, crow dipper, croton seed, dioscorea root, ginkgo seed, wolfberry, horny goat weed, lily bulb, rhubarb, round cardamon fruit, thunder vine, trichosanthis root, strychnine tree seed, sweet wormwood, willow bark, astragalus, atractylodes, bupleurum, cinnamon twigs, cinnamon bark, coptis, hoelen, ephedra sinica, peony, rehmannia, rhubarb, salvia, margarita, realgar, fructus gardeniae, borneolum syntheticum, carapax et plastrum testudinis, loquat leaves, pomelo leaves, poria cocos, platycodon grandiflorum, polygala root, rhizoma pinellinae, korean mint, chinese alangium root, chinese anemone, anisodus tanguticus, marlberry, tatar aster, tea plant, cannabis, safflower, cassia, velvet leaf, chinese goldthread, fumewort, purging croton, lilac daphne, devil's trumpet, jimson weed, noble dendrobium, chinese quinine, chinese ephedra, hardy rubber tree, peking spurge, flueggea suffruticosa, weeping forsythia, gentiana loureiroi, chinese honeylocust, licorice, chaulmoogra tree, purple holly, szechwan lovage, amur cork tree, chinese arborvitae, golden larch, naked rue, kudzu, sarpagandha, chinese foxglove, chinese rhubarb, rhododendron tsinghaiense, costus, chinese magnolia vine, baikal skullcap, pagoda tree, chinese cucumber, stringbush, akebia quinata, arsenic trioxide, arsenolite, calomelas, chenpi, cinnabaris, clematis, curcuma, dalbergia odorifera, persicaria, polygonum, sparganium, zedoary, agkistrodon, arisaema cum bile, aspongopus, asteriscus pseudosciaenae, benzoinum, bombyx batryticatus, bulbus fritillariae cirrhosae, bulbus fritillariae hupehensis, bulbus fritillariae pallidiflorae, bulbus fritillariae thunbergii, bulbus fritillariae ussuriensis, bulbus lycoridis radiatae, cacumen platycladi, cacumen securinegae suffruticosae, cacumen tamaricis, calamina, calculus bovis, calculus equi, calyx seu fructus physalis, caulis akebiae, caulis ampelopsis brevipedunculae, caulis aristolochiae manshuriensis, caulis bambusae in taeniam, caulis clematidis armandii, caulis entadae, caulis erycibes, caulis et folium piperis hancei, caulis et folium schefflerae arboricolae, caulis euphorbiae antiquori, caulis fibraureae, caulis gneti, caulis hederae sinensis, caulis impatientis, caulis lonicerae, caulis mahoniae, caulis perillae, caulis piperis kadsurae, caulis polygoni multiflori, caulis sargentodoxae, caulis sinomenii, caulis spatholobi, caulis tinosporae, caulis trachelospermi, cera chinensis, colla corii asini, concha arcae, concha haliotidis, concha margaritifera usta, concha mauritiae arabicae, concha meretricis seu cyclinae, concretio silicea bambusae, cordyceps, corium erinacei seu hemiechianus, cornu bubali, cornu cervi, cornu cervi degelatinatum, cornu cervi pantotrichum, cornu saigae tataricae, cortex acanthopanacis, cortex ailanthi, cortex albiziae, cortex cinchonae, cortex dictamni, cortex eucommiae, cortex fraxini, cortex hibisci, cortex Ilicis rotundae, cortex illicii, cortex kadsurae radicis, cortex lycii, cortex magnoliae officinalis, cortex meliae, cortex mori, cortex moutan, cortex periplocae, cortex phellodendri, cortex pseudolaricis, cortex schefflerae octophyllae, cortex ulmi parvifoliae, crinis carbonisatus, eupolyphaga seu steleophaga, faeces leporis, faeces trogopterori, faeces vespertilionis, flos albiziae, flos buddlejae, flos campsis, flos celosiae cristatae, flos chimonanthi praecocis, flos eriocauli, flos farfarae, flos genkwa, flos hibisci rosae-sinensis, fibs inulae, flos jasmini, flos magnoliae, flos magnoliae officinalis, flos rosae chinensis, fluoritum, folium aconiti kusnezoffii, folium apocyni veneti, folium artemisiae argyi, folium callicarpae formosanae, folium et cacumen breyniae fruticosae, folium et cacumen murrayae, folium et ramulus evodiae, folium ginkgo, folium ginseng, folium glochidii eriocarpi, folium hibisci mutabilis, folium ilicis cornutae, folium isatidis, folium lantanae camarae, folium nerii, folium photiniae, folium pini, folium polygoni tinctorii, folium psidii guajavae, folium psychotriae rubrae, folium pterocaryae stenopterae, folium pyrrosiae, folium rhododendri daurici, folium sauropi, folium sennae, folium viticis negundo, fructus akebiae, fructus alpiniae oxyphyllae, fructus amomi rotundus, fructus arctii, fructus aristolochiae, fructus aurantii, fructus aurantii immaturus, fructus broussonetiae, fructus bruceae, fructus carotae, fructus carpesii, fructus chebulae, fructus cnidii, fructus coriandri, fructus corni, fructus evodiae, fructus fici pumilae, fructus forsythiae, fructus galangae, fructus gleditsiae abnormalis, fructus kochiae, fructus leonuri, fructus ligustri lucidi, fructus liquidambaris, fructus litseae, fructus malvae, fructus nandinae domesticae, fructus phyllanthi, fructus piperis longi, fructus podophylli, fructus polygoni orientalis, fructus psoraleae, fructus quisqualis, fructus rosae laevigatae, fructus rubi, fructus sapindi mukorossi, fructus schisandrae, fructus seu herba physalis pubescentis, fructus sophorae, fructus terminaliae billericae, fructus toosendan, fructus tribuli, fructus trichosanthis, fructus tritici levis, fructus tsaoko, fructus ulmi macrocarpae, fructus viticis, fructus viticis negundo, fructus xanthii, galla chinensis, gekko swinhonis, gypsum fibrosum preparata, haematitum, halloysitum rubrum, herba abri, herba achyranthis asperae, herba aeschynomenes indicae, herba agastaches, herba aglaonematis, herba agrimoniae, herba andrographitis, herba antenoronis neofiliformis, herba ardisiae japonicae, herba aristolochiae, herba aristolochiae mollissimae, herba artemisiae annuae, herba artemisiae anomalae, herba artemisiae scopariae, herba asari, herba belladonnae, herba bidentis bipinnatae or herba bidentis pilosae, herba blumeae balsamiferae, herba bryophylli pinnati, herba casythae filiformis, herba catharanthi rosei, herba centellae, herba centipedae, herba chenopodii, herba cirsii, herba cissampelotis, herba cistanches, herba clinopodii, herba clinopodii gracilis, herba commelinae, herba corydalis bungeanae, herba crotalariae mucronatae, herba cymbopogonis, herba cynomorii, herba damnacanthi, herba dendrobii, herba desmodii styracifolii, herba dianthi, herba dichondrae repentis, herba duchesneae indicae, herba ecliptae, herba eleocharitis, herba elephantopi, herba eleusines indicae, herba emiliae, herba ephedrae, herba epimedii, herba epimeredis indicae, herba equiseti hiemalis, herba erodii or herba geranii, herba eupatorii, herba euphorbiae hirtae, herba euphorbiae humifusae, herba euphorbiae thymifoliae, herba gelsemii elegantis, herba gendarussae, herba geranii caroliniani, herba glechomae, herba gnaphalii affinis, herba gynostemmatis, herba hedyotidis chrysotrichae, herba hedyotidis corymbosae, herba houttuyniae, herba hyperici, herba hyperici erecti, herba hyperici japonici, herba inulae, herba junci setchuensis, herba kulanchoes laciniatae, herba leonuri, herba lespedezae cuneatae, herba lobeliae chinensis, herba lophatheri, herba lycopi, herba lycopodii, herba lysimachiae, herba malvastri coromandeliani, herba melastomatis candii, herba melastomatis dodecandri, herba orostachyos, herba paederiae, herba patriniae, herba pholidotae chinensis, herba phyllanthi urinariae, herba piperis sarmentosi, herba plantaginis, herba polygalae chinensis, herba polygalae japonicae, herba polygoni avicularis, herba polygoni chinensis, herba polygoni perfoliati, herba potentillae chinensis, herba potentillae discoloris, herba pouzolziae zeylanicae, herba pteridis multifidae, herba pteridis semipinnatae, herba pyrolae, herba rabdosiae lophanthoidis, herba ranunculi japonici, herba saginae japonicae, herba salviae chinensis, herba salviae plebeiae, herba sambuci chinensis, herba sarcandrae, herba saussureae involucratae, herba saxifragae, herba schizonepetae, herba scutellariae barbatae, herba sedi, herba selaginellae, herba selaginellae doederleinii, herba senecionis scandentis, herba setariae viridis, herba seu radix amaranrhi, herba seu radix cirsii japonici, herba sidae rhombifoliae, herba siegesbeckiae, herba solani lyrati, herba solani nigri, herba solidaginis, herba speranskiae tuberculatae, herba spirodelae, herba stephaniae longae, herba swertiae mileensis, herba taxilli, herba verbenae, herba veronicae, herba veronicastri, herba violae, herba visci, herba wedeliae, herba zephyranthis candidae, hirudo, indigo naturalis, lapis chloriti, lapis micae aureus, lasiosphaera seu calvatia, lignum dalbergiae odoriferae, lignum pini nodi, limonitum, medulla junci, medulla stachyuri or medulla helwingiae, medulla tetrapanacis, moschus, myrrha, natrii sulfas, natrii sulfas exsiccatus, nidus vespae, nux prinsepiae, oleum linderae, olibanum, omphalia, ootheca mantidis, ophicalcitum, pericarpium arecae, pericarpium trichosanthis, periostracum cicadae, petiolus trachycarpi, pollen pini, pollen typhae, polyporus, processed fructus crotonis, processed radix aconiti, processed radix aconiti kusnezoffii, processed radix aconiti lateralis, processed radix euphorbiae fischerianae, radix euphorbiae ebracteolatae, or radix stellerae, processed radix kansui, processed resina garciniae morellae, processed rhizoma arisaematis, processed rhizoma pinelliae, processed rhizoma typhonii or radix aconiti corean, processed semen crotonis, processed semen euphorbiae, processed semen hyoscyami, processed semen strychni, pseudobulbus cremastrae seu pleiones, pumex, pyrolusitum, radix acanthopanacis senticosi, radix acanthopanacis trifoliati, radix achyranthis bidentatae, radix adenophorae, radix aerio fici microcarpae, radix ampelopsis, radix angelicae citriodorae, radix angelicae pubescentis, radix angelicae sinensis, radix anisodi tangutici, radix ardisiae crenatae, radix aristolochiae, radix aristolochiae fangchi, radix aristolochiae heterophyllae, radix arnebiae or radix lithospermi, radix asparagi, radix asteris, radix astragali, radix aucklandiae, radix bauhiniae hupehanae, radix boehmeriae, radix bupleuri, radix celastri orbiculati, radix changii, radix chloranthi serrati, radix clematidis, radix clerodendri philippini, radix cocculi trilobi, radix codonopsis, radix crotonis crassifolii, radix curcumae, radix cyathulae, radix cynanchi atrati, radix cynanchi auriculati, radix cynanchi paniculati, radix cynoglossi amabilis, radix dichroae, radix dipsaci, radix echinopsis, radix ephedrae, radix et rhizoma nardostachyos, radix et rhizoma rhei, radix et rhizoma thalictri, radix et rhizoma veratri, radix eupatorii chinensis, radix euphorbiae pekinensis, radix gentianae, radix gentianae macrophyllae, radix ginseng, radix glehniae, radix hedysari, radix helicteris, radix hemerocallis, radix ilicis asprellae, radix ilicis pubescentis, radix inulae, radix isatidis, radix kadsurae coccineae, radix knoxiae, radix linderae, radix liriopes, radix mirabilis, radix morindae officinalis, radix notoginseng, radix ophiopogonis, radix oryzae glutinosae, radix paeoniae alba, radix paeoniae rubra, radix panacis quinquefolii, radix pandani tectorii, radix patriniae heterophyllae or radix patriniae scabrae, radix peucedani, radix physochlainae, radix phytolaccae, radix platycodi, radix plumbaginis zeylanicae, radix polygalae, radix polygoni multiflori, radix pseudostellariae, radix pterospermi heterophylli, radix pteroxygoni giraldii, radix puerariae, radix pulsatillae, radix ranunculus ternati, radix rauvolfiae, radix rehmanniae, radix rhapontici, radix rhodomyrti, radix rubiae, radix salviae miltiorrhizae, radix sanguisorbae, radix saposhnikoviae, radix scrophulariae, radix scutellariae, radix semiaquilegiae, radix seu caulis derridis trifoliatae, radix sophorae flavescentis, radix stellariae, radix stemonae, radix stephaniae tetrandrae, radix tinosporae, radix trichosanthis, radix tripterygii wilfordii, radix valerianae, radix vitis adstrictae, radix vladimiriae, radix wikstroemae, radix zanthoxyli, radix zanthoxyli avicennae, ramulus cinnamomi, ramulus et folium mussaendae pubescentis, ramulus et folium phyllodii pulchelli, ramulus et folium picrasmae, ramulus euonymi, ramulus mori, ramulus sambuci williamsii, ramulus uncariae cum uncis, receptaculum nelumbinis, resina draconis, resina ferulaen, resina liquidambaris, resina toxicodendri, rhizoma acori tatarinowii, rhizoma alismatis, rhizoma anemarrhenae, rhizoma anemones altaicae, rhizoma anemones raddeanae, rhizoma ardisiae gigantifoliae, rhizoma atractylodis, rhizoma atractylodis macrocephalae, rhizoma belamcandae, rhizoma bistortae, rhizoma blechni, rhizoma bletillae, rhizoma bolbostemmae, rhizoma chuanxiong, rhizoma cibotii, rhizoma cimicifugae, rhizoma coptidis, rhizoma corydalis, rhizoma corydalis decumbentis, rhizoma curculiginis, rhizoma curcumae, rhizoma curcumae longae, rhizoma cynanchi stauntonii, rhizoma cyperi, rhizoma cyrtomii, rhizoma dioscoreae bulbiferae, rhizoma dioscoreae cirrhosae, rhizoma dioscoreae hypoglaucae, rhizoma dioscoreae nipponicae, rhizoma dioscoreae septemlobae, rhizoma dioscoreae tokoro, rhizoma drynariae, rhizoma dryopteris crassirhizomae, rhizoma et radix baphicacanthis cusiae, rhizoma et radix notopterygii, rhizoma gastrodiae, rhizoma homalomenae, rhizoma ligustici, rhizoma matteucciae, rhizoma menispermi, rhizoma osmundae, rhizoma panacis japonici, rhizoma panacis majoris, rhizoma paridis, rhizoma picrorhizae, rhizoma polygonati, rhizoma polygoni cuspidati, rhizoma saururi or herba saururi, rhizoma smilacis chinensis, rhizoma smilacis glabrae, rhizoma sparganii, rhizoma wenyujin concisa, rhizoma woodwardiae, sal ammoniacus, scolopendra, scorpio, semen abri precatorii, semen abutili, semen aesculi, semen allii tuberosi, semen alpiniae katsumadai, semen arecae, semen astragali complanati, semen cassiae, semen cassiae occidentalis, semen celosiae, semen cuscutae, semen hoveniae, semen hydnocarpi, semen impatientis, semen lepidii or semen descurainiae, semen lini, semen momordicae, semen myristicae, semen nigellae, semen oroxyli, semen persicae, semen pharbitidis, semen plantaginis, semen platycladi, semen ricini, semen sinapis, semen sterculiae lychnophorae, semen trichosanthis, semen trigonellae, semen vaccariae, spica prunellae, spina gleditsiae, spongilla, spora lygodii, stamen nelumbinis, stigma croci, styrax, tabanus, and tremolitum.

At least one ingredient comprising at least one nutritional ingredient selected from group consisting of vitamin, dietary mineral, amino acid, and fatty acid. The vitamin includes vitamin A, vitamin B1, vitamin B2, vitamin B3, vitamin B5, vitamin B6, vitamin B7, vitamin B9, vitamin B12, vitamin C, vitamin D, vitamin E, and vitamin K. The dietary mineral includes calcium, phosphorus, potassium, sulfur, sodium, chlorine, magnesium, iron, cobalt, copper, zinc, molybdenum, iodine, selenium, manganese, nickle, chromium, fluorine, boron, and strontium. The amino acid includes histidine, isoleucine, leucine, lysine, methionine, cysteine, phenylalanine, tyrosine, threonine, tryptophan, and valine. The fatty acid includes alpha-linoleic acid, and linoleic acid.

At least one ingredient comprising at least one health ingredient selected from group consisting of medication, drug, and prescription drug, including caffeine.

At least one ingredient comprising at least one cleansing ingredient selected from group consisting of soap, detergent, deodorant, toothpaste, mouthwash, alcohol, camphor, menthol, phenol, eucalyptus, baking soda, fluoride, triclosan, sulfur, iodine, bleach, Epsom salt, salt, salicylic acid, benzoyl peroxide, resorcinol, hydrogen peroxide, chlorine, ammonia, vinegar, citric acid, lye, triclocarban, chlorhexidine gluconate, chloroxylenol, plant oil, vegetable oil, organic oil, jojoba oil, aloe vera, mineral oil, plant wax, animal wax, beeswax, carnauba wax, paraffin wax, propolis, petroleum jelly, potassium alum, ammonium alum, aluminum zirconium tetrachlorohydrex gly, activated charcoal, zeolite, silica gel, oxidizing agent, air sanitizer, antiseptic, deodorant, and disinfectant.

At least one ingredient comprising at least one food additive selected from group consisting of acid, acidity regulator, alcohol, anticaking agent, antifoaming agent, antioxidant, binding agent, bulking agent, coagulation agent, food coloring, color retention agent, emulsifier, flavor enhancer, flour treatment agent, gelling agent, glazing agent, humectant, leavening agent, tracer gas, preservative, stabilizer, sweetener, tenderizer, and thickener.

The ingredient particles are created by grinding of at least one ingredient into small particles.

The ingredient particles can be brewed in boiling water for about ½ to 20 minutes before consumption of resulting ingredient drink, which can be cooled with refrigeration.

The ingredient particles are created by method of boiling of at least one ingredient into ingredient drink; and by method selected from group consisting of drying of ingredient drink into soluble powder, freeze drying of ingredient drink into granules, and spray drying of ingredient drink into granules.

The ingredient particles can be dissolved in hot water for consumption of resulting ingredient drink, which can be cooled with refrigeration.

The ingredient particles can be used as seasoning powder.

The ingredient particles can be enclosed in small bottle of sprinkler having small openings at top of sprinkler for convenience and ease of use seasoning food and drink in suitable amounts.

The ingredient bag is created as porous bag containing the ingredient particles. The porous bag having material selected from group consisting of paper, cloth, rubber, and bio-plastic.

The ingredient bag can be brewed in boiling water for about ½ to 20 minutes before consumption of resulting ingredient drink, which can be cooled with refrigeration.

The ingredient dried pieces are created by method selected from group consisting of cut into thin slices, cut into small pieces, grind into small particles, grind into powder, mold into spherical shape, mold into geometrical shape, and mold into non geometrical shape; and by method selected from group consisting of sun drying, drying by heat, deep frying, baking, steaming, boiling, roasting, grilling, and frying.

The ingredient dried pieces comprising addition of at least one flavoring ingredient selected from group consisting of salt, vinegar, sugar, honey, syrup, ginger, garlic, chocolate, coffee, tea, caramel, butterscotch, peanut butter, butter, margarine, cheese, cream cheese, cottage cheese, mozzarella, mascarpone, ricotta, cream, sour cream, milk, ice cream, yogurt, gelato, sherbet, vegetable gum, vegetable starch, vegetable shortening, fish oil, fish liver oil, plant wax, animal wax, beeswax, carnauba wax, royal jelly, glycerol, egg, seafood, meat, soup stock, lactase, yeast, propolis, quinine, and malt. The at least one flavoring ingredient is in form selected from group consisting of skin, skinned, unskinned, unshelled, cut, uncut, extract, juice, sauce, liquid, oil, gel, jelly, wax, cream, paste, powder, thin layer, small particles, and small pieces. The at least one flavoring ingredient can be uncooked, and cooked by method selected from group consisting of deep frying, sun drying, roasting, baking, steaming, boiling, grilling, and frying.

The ingredient dried pieces are covered by at least one outer coating selected from group consisting of having an outer layer of casing selected from group consisting of intestine, gelatin, and cellulose; and comprising at least one coating selected from group consisting of bread crumbs, beer batter, egg batter, cornmeal batter, milk batter, yogurt batter, tea batter, coffee batter, malt batter, chocolate batter, honey batter, syrup batter, caramel batter, butterscotch batter, peanut butter batter, butter batter, margarine batter, cheese batter, cereal grain batter, nut batter, seed batter, bean batter, vegetable batter, plant batter, herb batter, fruit batter, wine batter, liquor batter, alcoholic batter, soft drink batter, soup batter, plant wax, animal wax, beeswax, carnauba wax, donut, roll, pie, pastry, waffle, crepe, pancake, pita, tortilla, bread, taco, biscuit, cookie, cake, jelly, royal jelly, cheese, cream cheese, cottage cheese, mozzarella, mascarpone, ricotta, butter, margarine, pudding, custard, candy, ice cream, gelato, sherbet, yogurt, chocolate, fudge, and at least one ingredient.

The ingredient dried pieces can be used as snack food.

The clothing and covering including T-shirt, panty, bra, brief, boxer, short, underpant, long john, bikini, thong, G-string, jacket, parka, coat, blazer, trench, shirt, sleeveless shirt, polo shirt, cardigan, sweater, sweatshirt, hoodie, pant, trouser, jean, suit, tuxedo, vest, tie, bow tie, handkerchief, scarf, muffler, dress, skirt, short, capri, gown, cape, cloak, robe, mantle, poncho, shawl, shroud, stole, face mask, veil, hat, cap, helmet, flag, badge, patch, handband, wristband, armband, elbowband, ankleband, kneeband, footband, legband, waistband, glove, sock, panty hose, legging, tight, leotard, night gown, lingerie, apron, bottom, top, towel, paper towel, kitchen towel, bathroom towel, dishcloth, dustcloth, air filter, cloth, bandage, pad, belt, bottle, feeding bottle, spray bottle, pump bottle, container, paper container, plastic container, metal container, thermal container, pressurized container, food container, drink container, cosmetics container, fragrance container, moisturizer container, cleanser container, soap dispenser, tube, box, paper box, plastic box, metal box, thermal box, lunchbox, jewelry box, watch box, eyewear box, wallet, purse, key holder, backpack, handbag, duffel bag, satchel bag, messenger bag, sling bag, golfbag, laptop bag, bag, tote, sack, suitcase, briefcase, blanket, quilt, pillow, cushion, comforter, duvet cover, sham, pillow case, bed sheet, mattress cover, mattress pad, seating pad, tablecloth, linen, bedding, furniture covering, computer covering, keyboard cover, cell phone covering, mousepad, pad, carpet, rug, mat, doormat, bathmat, placemat, car mat, cover, car cover, vehicle cover, car seat cover, sofa cover, couch cover, cushion cover, paint, wall, ceiling, roof, floor, wall cover, ceiling cover, floor cover, shade, shield, blind, door, plastic door, glass door, metal door, window, plastic window, glass window, metal window, car window, window shade, window blind, shutter, screen, window screen, TV screen, computer screen, cell phone screen, curtain, shower curtain, umbrella, eyeglass, sunglass, tent, sleeping bag, punching bag, sandbag, air bag, tea bag, popcorn bag, lunch bag, paper bag, plastic bag, grocery bag, shopping bag, gunny sack, thermal bag, body bag, burn bag, money bag, mail bag, antistatic bag, bulk bag, disposable bag, reusable bag, garbage bag, airsickness bag, zipper bag, drawstring bag, string bag, diaper bag, mosquito net, car cover, shoe, boot, sandal, slipper, sneaker, skate, lens, eyeglass, sunglass, uniform, armor, ballistic vest, bulletproof vest, bombsuit, life jacket, firesuit, protective suit, spacesuit, pressure suit, baselayer, underwear, lounge wear, brassiere, hosiery, daywear, evening wear, formal wear, bridal wear, outerwear, earwear, eyewear, headwear, footwear, dance suit, swimsuit, diving suit, track suit, skiwear, snowwear, rainwear, beach wear, activewear, sportswear, vehicle, and building.

At least one ingredient comprising medicine can be applied to contraceptive so that medicine can be applied directly to sex organ instead of taken by mouth which causes delay in response. At least one ingredient can be applied to medical device so that medicine can be applied directly to specific part of body instead of taken by mouth which causes delay in response. The contraceptive including male condom, female condom, sponge, diaphragm, cervical cap, lubricant, intrauterine device, and intravaginal device. The medicine including aphrodisiac, birth control medicine, medicine for sexually transmitted diseases, medicine for vagina, medicine for colon, and medicine for specific part of body. The medicine can be applied as imbedded, outer coating, and inner coating. The intravaginal device including intravaginal ring, and intravaginal cylindrical tube. The intravaginal cylindrical tube is selected from group consisting of having openings at both ends, having closed end at side of uterus, having length of vagina, having length less than length of vagina, having length of about 1 to 16 inches, having opening at side of vagina opening, having round opening at side of vagina opening, having flexible ring surrounding opening for holding in place, having flexible ring at closed end at side of uterus for holding in place, having flexible ring about 0.1 to 10 millimeters in thickness, having flexible ring shape of torus, having flexible ring shape of hollow disc about 2 to 10 millimeters in width, having a soft layer of cylindrical tube, having a molded layer of cylindrical tube, having a thin layer of cylindrical tube of about 0.01 to 5 millimeters, having small and large sizes, having silicone based lubricant as outer coating, having silicone base lubricant as inner coating, having silicone base lubricant as outer and inner coating, having texture as studded, having texture as ribbed, having texture on outside, having texture on inside, having texture on outside and inside, made of silicone, made of elastomer, made of polyethylene, made of polyurethane, made of nitrile, made of plastic, made of latex, and made of intestine. The intravaginal cylindrical tube can be used for insertion into anus.

At least one ingredient comprising vitamin C can be applied to food container so that food is kept fresh for a longer period of time. The vitamin C is equivalent to increased acidity and low pH level. The vitamin C is applied by method selected from group consisting of imbedded, coated on outside, coated on inside, and coated on outside and inside. The food container is selected from group consisting of paper container, plastic container, glass container, ceramic container, metal container, kitchen cabinet, and refrigerator.

Ingredient for consumption and application having packaging as individual package and multiple package The packaging is in air tight container The packaging is selected from group consisting of paper, plastic, metal, and glass.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. Ingredient for consumption and application comprising:
at least one ingredient;
wherein said at least one ingredient having nutrients including carbohydrates, vitamins, minerals, and antioxidants;
wherein said at least one ingredient having medicinal properties;
wherein said at least one ingredient having cleansing properties;
wherein said at least one ingredient is in form selected from group consisting of skin, skinned, unskinned, unshelled, cut, uncut, extract, juice, sauce, liquid, oil, gel, jelly, wax, cream, paste, powder, thin layer, small particles, small pieces, and large pieces;
wherein said at least one ingredient can be uncooked;
wherein said at least one ingredient can be cooked by method selected from group consisting of deep frying, sun drying, roasting, baking, steaming, boiling, grilling, and frying;
wherein said at least one ingredient can be added to various fabric including waterproof fabric, synthetic fabric, and natural fabric;
wherein said at least one ingredient can be added to various fabric by method selected from group consisting of imbedded, coated, sewn, and glued; and
wherein said at least one ingredient can be added to various medium including solid, liquid, gas, and vacuum.

2. Ingredient for consumption and application of claim 1 wherein said at least one ingredient for consumption can be delivered in form selected from group consisting of ingredient particles, ingredient bag, ingredient dried pieces, as mixed in for food and drink, as topping for food and drink, as mixed in for condiment, as mixed in for organic matter, as coating for organic matter, as mixed in for inorganic matter, and as coating for inorganic matter.

3. Ingredient for consumption and application of claim 1 wherein said at least one ingredient for application can be delivered in form selected from group consisting of clothing, covering, porous container, candle, fragrance, cosmetics, cleanser, moisturizer, lotion, cream, gel, foam, ointment, wax, oil, sunscreen, bath oil, nasal spray, transdermal patch, astringent, face mask, deodorant, antiperspirant, talcum powder, contraceptive, condom, contraceptive sponge, sponge, hair brush, face brush, body brush, dental floss, mouthwash, toothpaste, toothbrush, shampoo, conditioner, soap, detergent, tissue paper, sanitary pad, sanitary napkin, panty liner, tampon, incontinence pad, diaper, underarm liner, sofa, chair, mattress, pillow, cushion, clothes hanger, toy, ornament, jewelry, musical instrument, photo frame, picture frame, vase, flower pot, glassware, dinnerware, tableware, cookware, furniture, car seat, motorcycle seat, bike seat, cleaning device, medical device, wheeled device, exercise equipment, toilet, sink, bathtub, shower, sauna, countertop, kitchen appliance, and home appliance.

4. Ingredient for consumption and application of claim 1 wherein said at least one ingredient comprising at least one plant ingredient selected from group consisting of fruit, plant, plant oil, herb, tea, root, flower, flower bud, pollen, leaf, branch, bark, stem, stem shoot, bulb, nut, seed, bean, cereal grain, fruit skin, vegetable skin, vegetable, vegetable oil, and vegetable fat, including hop, menthol, coryceps, echinacea, elder, horehound, mallow, sage, thyme, cowslip, burnet, yarrow, marsh mallow, lady's mantle, speedwell, plantain, linden flower, wild thyme, hyssop, eucalyptus, sea buckthorn, elder blossom, verbena, lemon balm, clove, coriander, cumin, basil, dill, rosemary, parsley, fennel, oregano, allspice, catnip, wormwood, musk, chili, horseradish, turmeric, mustard, curry, wasabi, mountain mint, peppermint, spearmint, wintergreen, cinnamon, licorice, rose, jasmine, chamomile, chrysanthemum, lavender, acacia, safflower, vanilla, asafoetida, balsam, bay, buchu, birch, camphor, cannabis, caraway, calamus, cistus, citronella, clary sage, costus, cubeb, cypress, cypriol, davana, elecampane, fenugreek, fir, frankincense, galbanum, geranium, goldenrod, henna, helichrysum, hickory, juniper, ledum, litsea cubeba, marjoram, melaleuca, mountain savory, mugwort, myrrh, myrtle, neroli, nutmeg, orris, palo santo, petitgrain, pine, ravensara, red cedar, rosewood, sage, sassafras, schisandra, spikenard, spruce, tea tree, tsuga, valerian, yarrow, ylang ylang, zedoary, benzoin, copaiba, manuka, bergamot, patchouli, lilium, ambergris, gardenia, oakmoss, labdanum, coumarin, iris, water lily, ivy, amber, carnation, vetiver, cardamom, freesia, cyclamen, muguet, gardenia, tuberose, tonka bean, agarwood, cedarwood, sandalwood, alfafa, apple, orange, lemon, lime, grapefruit, grape, guava, plum, peach, pear, blackberry, blueberry, strawberry, cranberry, raspberry, cherry, mango, pineapple, pomegranate, papaya, watermelon, rockmelon, hamimelon, melon, honeydew, coconut, durian, passion fruit, banana, fig, apricot, mandarin, blackcurrant, starfruit, kiwi, pomelo, lychee, loquat, persimmon, tangerine, mangosteen, noni, sugar apple, mulberry, elderberry, gooseberry, lingonberry, juniper berry, jackfruit, dragonfruit, abiu, acai, acerola, ackee, cherry orange, moringa, ambarella, araza, arhat, atemoya, atherton raspberry, babaco, bacupari, bacuri, bael, barbadine, beach plum, batuan, bearberry, bignay, bilberry, bilimbi, biriba, black apple, black cherry, black raspberry, black mulberry, black sapote, blood orange, bolwarra, boquila, blue tongue, breadfruit, broad leaf bramble, buddha's hand, buffaloberry, brush cherry, burdekin plum, bush tomato, button mangosteen, caimito, calabash tree, calamondin, calamansi, camucamu, canary melon, canistel, cape gooseberry, capuli cherry, carambola, cardon, carob, cashew apple, cassabanana, cattley guava, cawesh, cedar bay cherry, ceriman, cereus peruvianus, charichuelo, chayote, che, chempedak, chenet, cherimoya, cherry ballart, chokeberry, chokecherry, chupa chupa, citron, clementine, cloudberry, cluster fig, cocky apple, coco plum, cocona, cocoplum, coffee cherry, common apple berry, conkerberry, cornelian cherry, crowberry, cupuacu, currant, curry leaf apple, custard apple, elephant apple, key apple, damson plum, date, date plum, davidson's plum, dead man's finger, desert banana, desert fig, desert lime, dodder laurel, doubah, double coconut, duku, eastern may hawthorn, emu apple, emu berry, false mastic, feijoa, fibrous satinash, finger lime, florida strangler fig, gac, galia melon, gambooge, genip, giant granadilla, otaheite gooseberry, goumi, governor's plum, montessa granadilla, yellow granadilla, greengage, grumichama, guanabana, guarana, cattley guava, pineapple guava, purple guava, strawberry guava, guavaberry, hackberry, hairless rambutan, hardy kiwi, hawthorn, hog plum, honeysuckle, horned melon, huckleberry, huito, ice cream bean, llama, lllawarra plum, imbe, jabuticaba, jambul, jatoba, jelly palm, jocote, jujube, kabosu, kaffir lime, kahikatea, kakadu lime, kakadu plum, kapok, karkalla, karonda, kei apple, kepel fruit, keule, key lime, kitembilla, korlan, kumquat, kutjera, kwai muk, lady apple, lakoocha, langsat, lanzone, lapsi, lardizabala, lemon aspen, leucaena, lillypilly, little gooseberry tree, limequat, loganberry, longan, lucuma, ma praang, mabolo, madrono, malabar plum, mamey sapote, mamoncillo, mangaba, manoao, maqui, marang, manila, mayapple, malay apple, mammee apple, maypop, medlar, melinjo, melon pear, midyim, miracle fruit, mock strawberry, purple mombin, red mombin, yellow mombin, monstera, mora de castilla, morinda, morinda citrifolia, mortiño, mountain pepper, mountain soursop, wild plum, mundu, muntries, muscadine, muskmelon, naartjie, nageia, nance, nannyberry, naranjilla, neem, nere, nonda plum, nungu, oil palm, oregan grape, oroblanco, otaheite gooseberry, palmyra palm, pawpaw, peachpalm, peanut butter fruit, pepino, pequi, peumo, phalsa, pigeon pea, pigeon plum, pigface, pindo palm, pineapple guava, pitomba, pois doux, pommecythere, pommerac, prickly pear, prune, pulasan, pummelo, pupunha, purple apple berry, purple granadilla, quandong, queensland ebony, quince, raisin tree, rambutan, red granadilla, red mulberry, rhubarb, riberry, rimu, rollinia, rose apple, rose hip, rose leaf bramble, rose myrtle, rowan, safou, sageretia, saguaro, salak, salal, salmonberry, sandpaper fig, santol, mamey, sapodilla, fibrous satinash, saskatoonberry, saw palmetto, sea buckthorn, serviceberry, shipova, small leaf tamarind, snow berry, soncoya, servicetree, soursop, southern crabapple, spanish lime, spanish tamarind, spiny monkey orange, star apple, carambola, strawberry guava, ugni, strawberry pear, surinam cherry, sweet apple berry, sweet ganadilla, sweet lemon, sweet orange, sweet pepper, sweetsop, sycamore fig, tamarillo, tamarind, monkey tamarind, velvet tamarind, tanjong, texas persimmon, thimbleberry, totara, toyon, wampee, water apple, wax apple, wax jambu, acerola, white aspen, white mulberry, white sapote, wild grape, wild orange, wild peach, wild plum, wild mangosteen, wineberry, wolfberry, wongi, wood apple, yangmei, yellow plum, yew, youngberry, zig zag vine, ahipa, arracacha, bamboo shoot, beetroot, burdock, broadleaf arrowhead, camas, canna, carrot, cassava, celeriac, chinese artichoke, daikon, earthnut pea, elephant foot yam, ensete, galangal, ginger, ginseng, hamberg parsley, lotus root, jerusalem artichoke, jicama, mashua, parsley root, parsnip, pignut, potato, prairie turnip, radish, rutabaga, salsify, scorzonera, skirret, swede, sweet potato, taro, ti, tigernut, turnip, ulluco, wasabi, water caltrop, water chestnut, yalcon, yam, avocado, bell pepper, bitter melon, bitter gourd, breadfruit, chayote, cucumber, ivy gourd, eggplant, aubergine, brinjal, luffa, nopal, okra, pepper, pumpkin, squash, sweet corn, sweet pepper, tinda, tomatillo, tomato, west indian gherkin, winter melon, zucchini, courgette, artichoke, broccoli, caper, cauliflower, courgette flower, globe artichoke, squash blossom, asparagus, prussian asparagus, bamboo shoot, celery, leek, wild leek, lemongrass, rhubarb, cardoon, kurrat, galangal, amaranth, arugula, beet green, bok choy, borage, broccoli rabe, brussel sprout, cabbage, catsear, celery, celtuce, chard, chaya, chickweed, chicory, chinese mallow, chive, choi sum, collard green, common purslane, corn salad, cress, dandelion, endive, fat hen, fiddlehead, fluted pumpkin, garden rocket, golden samphire, good king henry, greater plantain, kai lan, kale, komatsuna, kuka, lagos bologi, lamb's lettuce, lamb's quarters, land cress, leek, lettuce, lizard's tail, malabar spinach, melokhia, miner's lettuce, mizuna green, mustard green, napa cabbage, new zealand spinach, orache, pak choy, paracress, pea, poke, radicchio, samphire, sculpit, stridolo, sea beet, sea kale, sierra leoni bologi, soko, sorrel, spinach, summer purslane, swiss chard, tatsoi, turnip green, watercress, water spinach, wheatgrass, winter purslane, yarrow, yao choy, cardoon, chive, florence fennel, elephant garlic, garlic, garlic chive, kohlrabi, onion, pearl onion, potato onion, spring onion, tree onion, welsh onion, quamash, scallion, shallot, rice, barley, oat, barley, rye, corn, wheat, buckwheat, millet, sorghum, spelt, quinoa, chicory, molasses, beet root, fonio, palmer's grass, teff, triticale, cattail, chia, flax, grain amaranth, canihua, pitseed goosefoot, wattleseed, almond, cashew, chestnut, coconut, hazelnut, macadamia, peanut, pecan, pine nut, pistachio, walnut, acorn, brazil nut, gingko nut, shea nut, beechnut, candlenut, kurrajong, mongongo, palm nut, karuka, red bopple nut, hickory, jacknut, pepea nut, yellow walnut, cycad, munya nut, monkey puzzle nut, paradise nut, colocynth, filbert, mamoncillo, maya nut, ogbono nut, water caltrop, juniper, podocarp, cempedak, coffee, egusi, fox nut, jackfruit, malabar gourd, nutmeg, breadnut, betel nut, cola nut, pili nut, olive, drumstick, okra, pumpkin seed, sesame seed, sunflower seed, lotus seed, hemp seed, green bean, red bean, azuki bean, black bean, back eyed bean, white bean, yellow bean, common bean, broad bean, pinto bean, soybean, lima bean, kidney bean, pea bean, potato bean, dolichos bean, moth bean, mung bean, ricebean, runner bean, tepary bean, urad bean, velvet bean, winged bean, yardlong bean, chickpea, pigeon pea, snap pea, snow pea, lentil, lupin, tarwi, carob, guar, horse gram, cocoa bean, coffee bean, bambara groundnut, cowpea, hyacinth bean, lablab, moringa oleifera, sterculia, yam bean, alfalfa, clover, mesquite, carob, tamarind, wisteria, aonori, arame, carola, caulerpa, dabberlocks, dulse, hijiki, kombu, laver, mozuku, nori, ogonori, sea grape, sea lettuce, sea weed, wakame, truffle, mushroom, bean sprout, acai, aloe vera, arnica, ashoka, asthma weed, astragalus, barberry, belladonna, bitter leaf, bitter orange, black cohosh, blessed thistle, cat's claw, cayenne, chaparral, chasteberry, cinchona, coffee senna, comfrey, digitalis, dong quai, ephedra, european mistletoe, evening primrose, fenugreek, feverfew, flaxseed, gingko, goldenseal, hoodia, horse chestnut, horsetail, jamaica dogwood, jojoba, kava, khat, konjac, kratom, kanna, licorice root, marigold, moringa oleifera, milk thistle, neem, opium poppy, purple coneflower, passion flower, red clover, syrian rue, st john's wort, saw palmetto, tea tree oil, thunder god vine, tulasi, turmeric, umckaloabo, valerian, white willow, yerba santa, ajwain, akudjura, alexanders, alkanet, alligator pepper, angelica, anise, anise hyssop, aniseed myrtle, annatto, apple mint, artemisia, asafoetida, asarabacca, avens, avocado leaf, barberry, bay leaf, bee balm, boldo, borage, black cardamom, blue fenugreek, caraway, cardamom, cassia, cayenne pepper, celery leaf, celery seed, chervil, chicory, cicely, cilantro, clary, costmary, cubeb pepper, cudweed, culantro, curry leaf, curry plant, dill seed, epazote, fenugreek, filé powder, fingerroot, galangal, galingale, ganoderma, golpar, grains of paradise, grains of selim, houttuynia cordata, huacatay, indonesian bay leaf, jimbu, kaffir lime leaves, kala zeera, kawakawa seeds, kencur, keluak, kinh gioi, kokam seed, korarima, koseret leaves, lemon myrtle, lemon ironbark, lemon verbena, leptotes bicolor, lesser calamint, lime flower, lovage, mahlab, marjoram, mastic, mountain horopito, musk mallow, nasturtium, nigella, njangsa, neem, olida, orris root, pandan flower, pandan leaf, paprika, paracress, peppermint gum leaf, perilla, pandanus amaryllifolius, quassia, ramsons, rice paddy herb, rue, saffron, salad burnet, salep, sassafras, savory, silphium, shiso, sorrel, spikenard, star anise, sumac, sweet woodruff, tarragon, voatsiperifery, water-pepper, wattleseed, wild betel, willow herb, wood avens, woodruff, abies balsamea, abronia fragrans, acer glabrum var. douglasii, acer glabrum var. glabrum, acer negundo, acer saccharinum, acer saccharum, actaea racemosa, actaea rubra, agrimonia gryposepala, alnus rhombifolia, alnus rubra, artemisia douglasiana, artemisia ludoviciana, arundinaria, asarum canadense, asclepias verticillata, baccharis sarothroides, balsamorhiza sagittata, baptisia australis, betula occidentalis, blephilia ciliata, bloodroot, calypso, cardamine diphylla, culophyllum, canothus integerrimus, canothus velutinus, cimaphila umbellata, caytonia virginica, ceome serrulata, cmmelina dianthifolia, cornus sericea, delphinium nudicaule, devil's club, echinacea, encelia farinosa, ephedra californica, equisetum hyemale, erigenia bulbosa, eriodictyon crassifolium, eryngium aquaticum, erythrina herbacea, eurybia macrophylla, gaultheria hispidula, gaultheria procumbens, gentiana villosa, geranium maculatum, goldenseal, gutierrezia microcephala, hamamelis virginiana, heracleum maximum, holodiscus discolor, holodiscus dumosus, hydrangea arborescens, hydrangea cinerea, ilex verticillata, iris missouriensis, jeffersonia diphylla, juniperus communis, juniperus scopulorum, krascheninnikovia lanata, larrea tridentata, lobelia, mahonia aquifolium, mahonia nervosa, mahonia repens, malosma, menispermum canadense, osmunda claytoniana, pectis papposa, persicaria amphibia, pinus strobus, pluchea sericea, podophyllum peltatum, populus tremuloides, prunus emarginata, prunus virginiana, ribes aureum, ribes divaricatum, ribes glandulosum, ribes laxiflorum, ribes oxyacanthoides, salvia apiana, salvia mellifera, trichostema lanatum, trichostema lanceolatum, umbellularia, viburnum prunifolium, virginia iris, yucca glauca, agaricus bisporus, agaricus subrufescens, agrocybe aegerita, auricularia auricula, auricularia polytricha, boletus badius, boletus edulis, coprinus comatus, cordyceps sinensis, flammulina velutipes, grifola frondosa, hypsizygus tessellatus, lentinula edodes, lignosus rhinocerus, morchella esculenta, phallus impudicus, phallus indusiatus, pholiota nameko, pleurotus citrinopileatus, pleurotus djamor, pleurotus eryngii, pleurotus ostreatus, sparassis crispa, tremella fuciformis, tremella mesenterica, tricholoma matsutake, ustilago maydis, volvariella volvacea, astraeus hygrometricus, cyathus stercoreus, cyathus striatus, fomes fomentarius, ganoderma lucidum, hydnellum peckii, inonotus obliquus, peziza vesiculosa, phellinus linteus, piptoporus betulinus, polyporus umbellatus, poria cocos, schizophyllum commune, taiwanofungus camphoratus, trametes gibbosa, trametes versicolor, xylaria hypoxylon, fungus, and mold.

5. Ingredient for consumption and application of claim 1 wherein said at least one ingredient comprising at least one chinese medicine selected from group consisting of snake, snake venom, gecko, scorpion, seahorse, beetle, centipede, leech, fungus, cammellia, cayenne, chinese cucumber, cinchona tree, xanthium, crow dipper, croton seed, dioscorea root, ginkgo seed, wolfberry, horny goat weed, lily bulb, rhubarb, round cardamon fruit, thunder vine, trichosanthis root, strychnine tree seed, sweet wormwood, willow bark, astragalus, atractylodes, bupleurum, cinnamon twigs, cinnamon bark, coptis, hoelen, ephedra sinica, peony, rehmannia, rhubarb, salvia, margarita, realgar, fructus gardeniae, borneolum syntheticum, carapax et plastrum testudinis, loquat leaves, pomelo leaves, poria cocos, platycodon grandiflorum, polygala root, rhizoma pinellinae, korean mint, chinese alangium root, chinese anemone, anisodus tanguticus, marlberry, tatar aster, tea plant, cannabis, safflower, cassia, velvet leaf, chinese goldthread, fumewort, purging croton, lilac daphne, devil's trumpet, jimson weed, noble dendrobium, chinese quinine, chinese ephedra, hardy rubber tree, peking spurge, flueggea suffruticosa, weeping forsythia, gentiana loureiroi, chinese honeylocust, licorice, chaulmoogra tree, purple holly, szechwan lovage, amur cork tree, chinese arborvitae, golden larch, naked rue, kudzu, sarpagandha, chinese foxglove, chinese rhubarb, rhododendron tsinghaiense, costus, chinese magnolia vine, baikal skullcap, pagoda tree, chinese cucumber, stringbush, akebia quinata, arsenic trioxide, arsenolite, calomelas, chenpi, cinnabaris, clematis, curcuma, dalbergia odorifera, persicaria, polygonum, sparganium, zedoary, agkistrodon, arisaema cum bile, aspongopus, asteriscus pseudosciaenae, benzoinum, bombyx batryticatus, bulbus fritillariae cirrhosae, bulbus fritillariae hupehensis, bulbus fritillariae pallidiflorae, bulbus fritillariae thunbergii, bulbus fritillariae ussuriensis, bulbus lycoridis radiatae, cacumen platycladi, cacumen securinegae suffruticosae, cacumen tamaricis, calamina, calculus bovis, calculus equi, calyx seu fructus physalis, caulis akebiae, caulis ampelopsis brevipedunculae, caulis aristolochiae manshuriensis, caulis bambusae in taeniam, caulis clematidis armandii, caulis entadae, caulis erycibes, caulis et folium piperis hancei, caulis et folium schefflerae arboricolae, caulis euphorbiae antiquori, caulis fibraureae, caulis gneti, caulis hederae sinensis, caulis impatientis, caulis lonicerae, caulis mahoniae, caulis perillae, caulis piperis kadsurae, caulis polygoni multiflori, caulis sargentodoxae, caulis sinomenii, caulis spatholobi, caulis tinosporae, caulis trachelospermi, cera chinensis, colla corii asini, concha arcae, concha haliotidis, concha margaritifera usta, concha mauritiae arabicae, concha meretricis seu cyclinae, concretio silicea bambusae, cordyceps, corium erinacei seu hemiechianus, cornu bubali, cornu cervi, cornu cervi degelatinatum, cornu cervi pantotrichum, cornu saigae tataricae, cortex acanthopanacis, cortex ailanthi, cortex albiziae, cortex cinchonae, cortex dictamni, cortex eucommiae, cortex fraxini, cortex hibisci, cortex Ilicis rotundae, cortex illicii, cortex kadsurae radicis, cortex lycii, cortex magnoliae officinalis, cortex meliae, cortex mori, cortex moutan, cortex periplocae, cortex phellodendri, cortex pseudolaricis, cortex schefflerae octophyllae, cortex ulmi parvifoliae, crinis carbonisatus, eupolyphaga seu steleophaga, faeces leporis, faeces trogopterori, faeces vespertilionis, flos albiziae, flos buddlejae, flos campsis, flos celosiae cristatae, flos chimonanthi praecocis, flos eriocauli, flos farfarae, flos genkwa, flos hibisci rosae-sinensis, flos inulae, flos jasmini, flos magnoliae, flos magnoliae officinalis, flos rosae chinensis, fluoritum, folium aconiti kusnezoffii, folium apocyni veneti, folium artemisiae argyi, folium callicarpae formosanae, folium et cacumen breyniae fruticosae, folium et cacumen murrayae, folium et ramulus evodiae, folium ginkgo, folium ginseng, folium glochidii eriocarpi, folium hibisci mutabilis, folium ilicis cornutae, folium isatidis, folium lantanae camarae, folium nerii, folium photiniae, folium pini, folium polygoni tinctorii, folium psidii guajavae, folium psychotriae rubrae, folium pterocaryae stenopterae, folium pyrrosiae, folium rhododendri daurici,folium sauropi, folium sennae, folium viticis negundo, fructus akebiae, fructus alpiniae oxyphyllae, fructus amomi rotundus, fructus arctii, fructus aristolochiae, fructus aurantii, fructus aurantii immaturus, fructus broussonetiae, fructus bruceae, fructus carotae, fructus carpesii, fructus chebulae, fructus cnidii, fructus coriandri, fructus corni, fructus evodiae, fructus fici pumilae, fructus forsythiae, fructus galangae, fructus gleditsiae abnormalis, fructus kochiae, fructus leonuri, fructus ligustri lucidi, fructus liquidambaris, fructus litseae, fructus malvae, fructus nandinae domesticae, fructus phyllanthi, fructus piperis longi, fructus podophylli, fructus polygoni orientalis, fructus psoraleae, fructus quisqualis, fructus rosae laevigatae, fructus rubi, fructus sapindi mukorossi, fructus schisandrae, fructus seu herba physalis pubescentis, fructus sophorae, fructus terminaliae billericae, fructus toosendan, fructus tribuli, fructus trichosanthis, fructus tritici levis, fructus tsaoko, fructus ulmi macrocarpae, fructus viticis, fructus viticis negundo, fructus xanthii, galla chinensis, gekko swinhonis, gypsum fibrosum preparata, haematitum, halloysitum rubrum, herba abri, herba achyranthis asperae, herba aeschynomenes indicae, herba agastaches, herba aglaonematis, herba agrimoniae, herba andrographitis, herba antenoronis neofiliformis, herba ardisiae japonicae, herba aristolochiae, herba aristolochiae mollissimae, herba artemisiae annuae, herba artemisiae anomalae, herba artemisiae scopariae, herba asari, herba belladonnae, herba bidentis bipinnatae or herba bidentis pilosae, herba blumeae balsamiferae, herba bryophylli pinnati, herba casythae filiformis, herba catharanthi rosei, herba centellae, herba centipedae, herba chenopodii, herba cirsii, herba cissampelotis, herba cistanches, herba clinopodii, herba clinopodii gracilis, herba commelinae, herba corydalis bungeanae, herba crotalariae mucronatae, herba cymbopogonis, herba cynomorii, herba damnacanthi, herba dendrobii, herba desmodii styracifolii, herba dianthi, herba dichondrae repentis, herba duchesneae indicae, herba ecliptae, herba eleocharitis, herba elephantopi, herba eleusines indicae, herba emiliae, herba ephedrae, herba epimedii, herba epimeredis indicae, herba equiseti hiemalis, herba erodii or herba geranii, herba eupatorii, herba euphorbiae hirtae, herba euphorbiae humifusae, herba euphorbiae thymifoliae, herba gelsemii elegantis, herba gendarussae, herba geranii caroliniani, herba glechomae, herba gnaphalii affinis, herba gynostemmatis, herba hedyotidis chrysotrichae, herba hedyotidis corymbosae, herba houttuyniae, herba hyperici, herba hyperici erecti, herba hyperici japonici, herba inulae, herba junci setchuensis, herba kulanchoes laciniatae, herba leonuri, herba lespedezae cuneatae, herba lobeliae chinensis, herba lophatheri, herba lycopi, herba lycopodii, herba lysimachiae, herba malvastri coromandeliani, herba melastomatis candii, herba melastomatis dodecandri, herba orostachyos, herba paederiae, herba patriniae, herba pholidotae chinensis, herba phyllanthi urinariae, herba piperis sarmentosi, herba plantaginis, herba polygalae chinensis, herba polygalae japonicae, herba polygoni avicularis, herba polygoni chinensis, herba polygoni perfoliati, herba potentillae chinensis, herba potentillae discoloris, herba pouzolziae zeylanicae, herba pteridis multifidae, herba pteridis semipinnatae, herba pyrolae, herba rabdosiae lophanthoidis, herba ranunculi japonici, herba saginae japonicae, herba salviae chinensis, herba salviae plebeiae, herba sambuci chinensis, herba sarcandrae, herba saussureae involucratae, herba saxifragae, herba schizonepetae, herba scutellariae barbatae, herba sedi, herba selaginellae, herba selaginellae doederleinii, herba senecionis scandentis, herba setariae viridis, herba seu radix amaranrhi, herba seu radix cirsii japonici, herba sidae rhombifoliae, herba siegesbeckiae, herba solani lyrati, herba solani nigri, herba solidaginis, herba speranskiae tuberculatae, herba spirodelae, herba stephaniae longae, herba swertiae mileensis, herba taxilli, herba verenae, herba veronicae, herba veronicastri, herba violae, herba visci, herba wedeliae, herba zephyranthis candidae, hirudo, indigo naturalis, lapis chloriti, lapis micae aureus, lasiosphaera seu calvatia, lignum dalbergiae odoriferae, lignum pini nodi, limonitum, medulla junci, medulla stachyuri or medulla helwingiae, medulla tetrapanacis, moschus, myrrha, natrii sulfas, natrii sulfas exsiccatus, nidus vespae, nux prinsepiae, oleum linderae, olibanum, omphalia, ootheca mantidis, ophicalcitum, pericarpium arecae, pericarpium trichosanthis, periostracum cicadae, petiolus trachycarpi, pollen pini, pollen typhae, polyporus, processed fructus crotonis, processed radix aconiti, processed radix aconiti kusnezoffii, processed radix aconiti lateralis, processed radix euphorbiae fischerianae, radix euphorbiae ebracteolatae, or radix stellerae, processed radix kansui, processed resina garciniae morellae, processed rhizoma arisaematis, processed rhizoma pinelliae, processed rhizoma typhonii or radix aconiti corean, processed semen crotonis, processed semen euphorbiae, processed semen hyoscyami, processed semen strychni, pseudobulbus cremastrae seu pleiones, pumex, pyrolusitum, radix acanthopanacis senticosi, radix acanthopanacis trifoliati, radix achyranthis bidentatae, radix adenophorae, radix aerio fici microcarpae, radix ampelopsis, radix angelicae citriodorae, radix angelicae pubescentis, radix angelicae sinensis, radix anisodi tangutici, radix ardisiae crenatae, radix aristolochiae, radix aristolochiae fangchi, radix aristolochiae heterophyllae, radix arnebiae or radix lithospermi, radix asparagi, radix asteris, radix astragali, radix aucklandiae, radix bauhiniae hupehanae, radix boehmeriae, radix bupleuri, radix celastri orbiculati, radix changii, radix chloranthi serrati, radix clematidis, radix clerodendri philippini, radix cocculi trilobi, radix codonopsis, radix crotonis crassifolii, radix curcumae, radix cyathulae, radix cynanchi atrati, radix cynanchi auriculati, radix cynanchi paniculati, radix cynoglossi amabilis, radix dichroae, radix dipsaci, radix echinopsis, radix ephedrae, radix et rhizoma nardostachyos, radix et rhizoma rhei, radix et rhizoma thalictri, radix et rhizoma veratri, radix eupatorii chinensis, radix euphorbiae pekinensis, radix gentianae, radix gentianae macrophyllae, radix ginseng, radix glehniae, radix hedysari, radix helicteris, radix hemerocallis, radix ilicis asprellae, radix ilicis pubescentis, radix inulae, radix isatidis, radix kadsurae coccineae, radix knoxiae, radix linderae, radix liriopes, radix mirabilis, radix morindae officinalis, radix notoginseng, radix ophiopogonis, radix oryzae glutinosae, radix paeoniae alba, radix paeoniae rubra, radix panacis quinquefolii, radix pandani tectorii, radix patriniae heterophyllae or radix patriniae scabrae, radix peucedani, radix physochlainae, radix phytolaccae, radix platycodi, radix plumbaginis zeylanicae, radix polygalae, radix polygoni multiflori, radix pseudostellariae, radix pterospermi heterophylli, radix pteroxygoni giraldii, radix puerariae, radix pulsatillae, radix ranunculus ternati, radix rauvolfiae, radix rehmanniae, radix rhapontici, radix rhodomyrti, radix rubiae, radix salviae miltiorrhizae, radix sanguisorbae, radix saposhnikoviae, radix scrophulariae, radix scutellariae, radix semiaquilegiae, radix seu caulis derridis trifoliatae, radix sophorae flavescentis, radix stellariae, radix stemonae, radix stephaniae tetrandrae, radix tinosporae, radix trichosanthis, radix tripterygii wilfordii, radix valerianae, radix vitis adstrictae, radix vladimiriae, radix wikstroemae, radix zanthoxyli, radix zanthoxyli avicennae, ramulus cinnamomi, ramulus et folium mussaendae pubescentis, ramulus et folium phyllodii pulchelli, ramulus et folium picrasmae, ramulus euonymi, ramulus mori, ramulus sambuci williamsii, ramulus uncariae cum uncis, receptaculum nelumbinis, resina draconis, resina ferulaen, resina liquidambaris, resina toxicodendri, rhizoma acori tatarinowii, rhizoma alismatis, rhizoma anemarrhenae, rhizoma anemones altaicae, rhizoma anemones raddeanae, rhizoma ardisiae gigantifoliae, rhizoma atractylodis, rhizoma atractylodis macrocephalae, rhizoma belamcandae, rhizoma bistortae, rhizoma blechni, rhizoma bletillae, rhizoma bolbostemmae, rhizoma chuanxiong, rhizoma cibotii, rhizoma cimicifugae, rhizoma coptidis, rhizoma corydalis, rhizoma corydalis decumbentis, rhizoma curculiginis, rhizoma curcumae, rhizoma curcumae longae, rhizoma cynanchi stauntonii, rhizoma cyperi, rhizoma cyrtomii, rhizoma dioscoreae bulbiferae, rhizoma dioscoreae cirrhosae, rhizoma dioscoreae hypoglaucae, rhizoma dioscoreae nipponicae, rhizoma dioscoreae septemlobae, rhizoma dioscoreae tokoro, rhizoma drynariae, rhizoma dryopteris crassirhizomae, rhizoma et radix baphicacanthis cusiae, rhizoma et radix notopterygii, rhizoma gastrodiae, rhizoma homalomenae, rhizoma ligustici, rhizoma matteucciae, rhizoma menispermi, rhizoma osmundae, rhizoma panacis japonici, rhizoma panacis majoris, rhizoma paridis, rhizoma picrorhizae, rhizoma polygonati, rhizoma polygoni cuspidati, rhizoma saururi or herba saururi, rhizoma smilacis chinensis, rhizoma smilacis glabrae, rhizoma sparganii, rhizoma wenyujin concisa, rhizoma woodwardiae, sal ammoniacus, scolopendra, scorpio, semen abri precatorii, semen abutili, semen aesculi, semen allii tuberosi, semen alpiniae katsumadai, semen arecae, semen astragali complanati, semen cassiae, semen cassiae occidentalis, semen celosiae, semen cuscutae, semen hoveniae, semen hydnocarpi, semen impatientis, semen lepidii or semen descurainiae, semen lini, semen momordicae, semen myristicae, semen nigellae, semen oroxyli, semen persicae, semen pharbitidis, semen plantaginis, semen platycladi, semen ricini, semen sinapis, semen sterculiae lychnophorae, semen trichosanthis, semen trigonellae, semen vaccariae, spica prunellae, spina gleditsiae, spongilla, spora lygodii, stamen nelumbinis, stigma croci, styrax, tabanus, and tremolitum.

6. Ingredient for consumption and application of claim 1 wherein said at least one ingredient comprising at least one nutritional ingredient selected from group consisting of vitamin, dietary mineral, amino acid, and fatty acid; said vitamin includes vitamin A, vitamin B1, vitamin B2, vitamin B3, vitamin B5, vitamin B6, vitamin B7, vitamin B9, vitamin B12, vitamin C, vitamin D, vitamin E, and vitamin K; said dietary mineral includes calcium, phosphorus, potassium, sulfur, sodium, chlorine, magnesium, iron, cobalt, copper, zinc, molybdenum, iodine, selenium, manganese, nickle, chromium, fluorine, boron, and strontium; said amino acid includes histidine, isoleucine, leucine, lysine, methionine, cysteine, phenylalanine, tyrosine, threonine, tryptophan, and valine; said fatty acid includes alpha-linoleic acid, and linoleic acid.

7. Ingredient for consumption and application of claim 1 wherein said at least one ingredient comprising at least one health ingredient selected from group consisting of medication, drug, and prescription drug, including caffeine.

8. Ingredient for consumption and application of claim 1 wherein said at least one ingredient comprising at least one cleansing ingredient selected from group consisting of soap, detergent, deodorant, toothpaste, mouthwash, alcohol, camphor, menthol, phenol, eucalyptus, baking soda, fluoride, triclosan, sulfur, iodine, bleach, Epsom salt, salt, salicylic acid, benzoyl peroxide, resorcinol, hydrogen peroxide, chlorine, ammonia, vinegar, citric acid, lye, triclocarban, chlorhexidine gluconate, chloroxylenol, plant oil, vegetable oil, organic oil, jojoba oil, aloe vera, mineral oil, plant wax, animal wax, beeswax, carnauba wax, paraffin wax, propolis, petroleum jelly, potassium alum, ammonium alum, aluminum zirconium tetrachlorohydrex gly, activated charcoal, zeolite, silica gel, oxidizing agent, air sanitizer, antiseptic, deodorant, and disinfectant.

9. Ingredient for consumption and application of claim 1 wherein said at least one ingredient comprising at least one food additive selected from group consisting of acid, acidity regulator, alcohol, anticaking agent, antifoaming agent, antioxidant, binding agent, bulking agent, coagulation agent, food coloring, color retention agent, emulsifier, flavor enhancer, flour treatment agent, gelling agent, glazing agent, humectant, leavening agent, tracer gas, preservative, stabilizer, sweetener, tenderizer, and thickener.

10. Ingredient for consumption and application of claim 2 wherein said ingredient particles are created by grinding of said at least one ingredient into small particles.

11. Ingredient for consumption and application of claim 2 wherein said ingredient particles can be brewed in boiling water for about ½ to 20 minutes before consumption of resulting ingredient drink, which can be cooled with refrigeration.

12. Ingredient for consumption and application of claim 2 wherein said ingredient particles are created by method of boiling of said at least one ingredient into ingredient drink; and by method selected from group consisting of drying of said ingredient drink into soluble powder, freeze drying of said ingredient drink into granules, and spray drying of said ingredient drink into granules.

13. Ingredient for consumption and application of claim 2 wherein said ingredient particles can be dissolved in hot water for consumption of resulting ingredient drink, which can be cooled with refrigeration.

14. Ingredient for consumption and application of claim 2 wherein said ingredient particles can be used as seasoning powder.

15. Ingredient for consumption and application of claim 2 wherein said ingredient particles can be enclosed in small bottle of sprinkler having small openings at top of sprinkler for convenience and ease of use seasoning food and drink in suitable amounts.

16. Ingredient for consumption and application of claim 2 wherein said ingredient bag is created as porous bag containing said ingredient particles; and said porous bag having material selected from group consisting of paper, cloth, rubber, and bio-plastic.

17. Ingredient for consumption and application of claim 2 wherein said ingredient bag can be brewed in boiling water for about ½ to 20 minutes before consumption of resulting ingredient drink, which can be cooled with refrigeration.

18. Ingredient for consumption and application of claim 2 wherein said ingredient dried pieces are created by method selected from group consisting of cut into thin slices, cut into small pieces, grind into small particles, grind into powder, mold into spherical shape, mold into geometrical shape, and mold into non geometrical shape; and by method selected from group consisting of sun drying, drying by heat, deep frying, baking, steaming, boiling, roasting, grilling, and frying.

19. Ingredient for consumption and application of claim 2 wherein said ingredient dried pieces comprising addition of at least one flavoring ingredient selected from group consisting of salt, vinegar, sugar, honey, syrup, ginger, garlic, chocolate, coffee, tea, caramel, butterscotch, peanut butter, butter, margarine, cheese, cream cheese, cottage cheese, mozzarella, mascarpone, ricotta, cream, sour cream, milk, ice cream, yogurt, gelato, sherbet, vegetable gum, vegetable starch, vegetable shortening, fish oil, fish liver oil, plant wax, animal wax, beeswax, carnauba wax, royal jelly, glycerol, egg, seafood, meat, soup stock, lactase, yeast, propolis, quinine, and malt; said at least one flavoring ingredient is in form selected from group consisting of skin, skinned, unskinned, unshelled, cut, uncut, extract, juice, sauce, liquid, oil, gel, jelly, wax, cream, paste, powder, thin layer, small particles, and small pieces; and said at least one flavoring ingredient can be uncooked, and cooked by method selected from group consisting of deep frying, sun drying, roasting, baking, steaming, boiling, grilling, and frying.

20. Ingredient for consumption and application of claim 2 wherein said ingredient dried pieces are covered by at least one outer coating selected from group consisting of having an outer layer of casing selected from group consisting of intestine, gelatin, and cellulose; and comprising at least one coating selected from group consisting of bread crumbs, beer batter, egg batter, cornmeal batter, milk batter, yogurt batter, tea batter, coffee batter, malt batter, chocolate batter, honey batter, syrup batter, caramel batter, butterscotch batter, peanut butter batter, butter batter, margarine batter, cheese batter, cereal grain batter, nut batter, seed batter, bean batter, vegetable batter, plant batter, herb batter, fruit batter, wine batter, liquor batter, alcoholic batter, soft drink batter, soup batter, plant wax, animal wax, beeswax, carnauba wax, donut, roll, pie, pastry, waffle, crepe, pancake, pita, tortilla, bread, taco, biscuit, cookie, cake, jelly, royal jelly, cheese, cream cheese, cottage cheese, mozzarella, mascarpone, ricotta, butter, margarine, pudding, custard, candy, ice cream, gelato, sherbet, yogurt, chocolate, fudge, and said at least one ingredient.

21. Ingredient for consumption and application of claim 2 wherein said ingredient dried pieces can be used as snack food.

22. Ingredient for consumption and application of claim 3 wherein said clothing and covering including T-shirt, panty, bra, brief, boxer, short, underpant, long john, bikini, thong, G-string, jacket, parka, coat, blazer, trench, shirt, sleeveless shirt, polo shirt, cardigan, sweater, sweatshirt, hoodie, pant, trouser, jean, suit, tuxedo, vest, tie, bow tie, handkerchief, scarf, muffler, dress, skirt, short, capri, gown, cape, cloak, robe, mantle, poncho, shawl, shroud, stole, face mask, veil, hat, cap, helmet, flag, badge, patch, handband, wristband, armband, elbowband, ankleband, kneeband, footband, legband, waistband, glove, sock, panty hose, legging, tight, leotard, night gown, lingerie, apron, bottom, top, towel, paper towel, kitchen towel, bathroom towel, dishcloth, dustcloth, air filter, cloth, bandage, pad, belt, bottle, feeding bottle, spray bottle, pump bottle, container, paper container, plastic container, metal container, thermal container, pressurized container, food container, drink container, cosmetics container, fragrance container, moisturizer container, cleanser container, soap dispenser, tube, box, paper box, plastic box, metal box, thermal box, lunchbox, jewelry box, watch box, eyewear box, wallet, purse, key holder, backpack, handbag, duffel bag, satchel bag, messenger bag, sling bag, golfbag, laptop bag, bag, tote, sack, suitcase, briefcase, blanket, quilt, pillow, cushion, comforter, duvet cover, sham, pillow case, bed sheet, mattress cover, mattress pad, seating pad, tablecloth, linen, bedding, furniture covering, computer covering, keyboard cover, cell phone covering, mousepad, pad, carpet, rug, mat, doormat, bathmat, placemat, car mat, cover, car cover, vehicle cover, car seat cover, sofa cover, couch cover, cushion cover, paint, wall, ceiling, roof, floor, wall cover, ceiling cover, floor cover, shade, shield, blind, door, plastic door, glass door, metal door, window, plastic window, glass window, metal window, car window, window shade, window blind, shutter, screen, window screen, TV screen, computer screen, cell phone screen, curtain, shower curtain, umbrella, eyeglass, sunglass, tent, sleeping bag, punching bag, sandbag, air bag, tea bag, popcorn bag, lunch bag, paper bag, plastic bag, grocery bag, shopping bag, gunny sack, thermal bag, body bag, burn bag, money bag, mail bag, antistatic bag, bulk bag, disposable bag, reusable bag, garbage bag, airsickness bag, zipper bag, drawstring bag, string bag, diaper bag, mosquito net, car cover, shoe, boot, sandal, slipper, sneaker, skate, lens, eyeglass, sunglass, uniform, armor, ballistic vest, bulletproof vest, bombsuit, life jacket, firesuit, protective suit, spacesuit, pressure suit, baselayer, underwear, lounge wear, brassiere, hosiery, daywear, evening wear, formal wear, bridal wear, outerwear, earwear, eyewear, headwear, footwear, dance suit, swimsuit, diving suit, track suit, skiwear, snowwear, rainwear, beach wear, activewear, sportswear, vehicle, and building.

23. Ingredient for consumption and application of claim 1 wherein said at least one ingredient comprising medicine can be applied to contraceptive so that medicine can be applied directly to sex organ instead of taken by mouth which causes delay in response; said at least one ingredient can be applied to medical device so that medicine can be applied directly to specific part of body instead of taken by mouth which causes delay in response; said contraceptive including male condom, female condom, sponge, diaphragm, cervical cap, lubricant, intrauterine device, and intravaginal device; said medicine including aphrodisiac, birth control medicine, medicine for sexually transmitted diseases, medicine for vagina, medicine for colon, and medicine for specific part of body; said medicine can be applied as imbedded, outer coating, and inner coating; said intravaginal device including intravaginal ring, and intravaginal cylindrical tube; said intravaginal cylindrical tube is selected from group consisting of having openings at both ends, having closed end at side of uterus, having length of vagina, having length less than length of vagina, having length of about 1 to 16 inches, having opening at side of vagina opening, having round opening at side of vagina opening, having flexible ring surrounding opening for holding in place, having flexible ring at closed end at side of uterus for holding in place, having flexible ring about 0.1 to 10 millimeters in thickness, having flexible ring shape of torus, having flexible ring shape of hollow disc about 2 to 10 millimeters in width, having a soft layer of cylindrical tube, having a molded layer of cylindrical tube, having a thin layer of cylindrical tube of about 0.01 to 5 millimeters, having small and large sizes, having silicone based lubricant as outer coating, having silicone base lubricant as inner coating, having silicone base lubricant as outer and inner coating, having texture as studded, having texture as ribbed, having texture on outside, having texture on inside, having texture on outside and inside, made of silicone, made of elastomer, made of polyethylene, made of polyurethane, made of nitrile, made of plastic, made of latex, and made of intestine; and said intravaginal cylindrical tube can be used for insertion into anus.

24. Ingredient for consumption and application of claim 1 wherein said at least one ingredient comprising vitamin C can be applied to food container so that food is kept fresh for a longer period of time; said vitamin C is equivalent to increased acidity and low pH level; said vitamin C is applied by method selected from group consisting of imbedded, coated on outside, coated on inside, and coated on outside and inside; and said food container is selected from group consisting of paper container, plastic container, glass container, ceramic container, metal container, kitchen cabinet, and refrigerator.

25. Ingredient for consumption and application of claim 1 having packaging as individual package and multiple package; said packaging is in air tight container; and said packaging is selected from group consisting of paper, plastic, metal, and glass.
